(19) United States
(12) Patent Application Publication
(10) Pub. No.: US 2022/0233773 A1
(43) Pub. Date: Jul. 28, 2022

(54) SYSTEMS AND METHODS FOR AUTOMATED INSULIN DELIVERY FOR DIABETES THERAPY

(71) Applicant: Tandem Diabetes Care, Inc., San Diego, CA (US)

(72) Inventors: Jose Ricardo Rueda, La Jolla, CA (US); Paul Harris, San Diego, CA (US); Thomas R. Ulrich, Oceanside, CA (US); Geoffrey A. Kruse, San Diego, CA (US)

(21) Appl. No.: 17/587,468

(22) Filed: Jan. 28, 2022

Related U.S. Application Data
(60) Provisional application No. 63/142,792, filed on Jan. 28, 2021.

(51) Int. Cl. A61M 5/172; A61M 5/142

(52) U.S. Cl. CPC A61M 5/1723; A61M 5/14248; A61M 2205/3553; A61M 2205/3561; A61M 2230/201

(57) ABSTRACT

Disclosed herein are systems and methods for automated insulin delivery that reduce a risk of hypoglycemia from automatically delivering correction boluses. Rather than automatically delivering a correction bolus when a current or future predicted glucose level of a user is over a high glucose threshold, the system can review additional factors to determine whether an automatic correction bolus is appropriate. For example, the system can be prevented from delivering an automatic correction bolus if the user's glucose levels are falling at greater than a predetermined rate and/or if a current glucose level is greater than a future predicted glucose level even if the user's current or predicted future glucose levels is over the high threshold.

### RELATED APPLICATION

This application claims the benefit of U.S. Provisional Application No. 63/142,792 filed Jan. 28, 2021, which is hereby fully incorporated herein by reference.

### FIELD OF THE INVENTION

The present invention relates generally to ambulatory infusion pumps and, more particularly, to operation of ambulatory infusion pumps in a closed-loop or semi-closed-loop fashion.

### BACKGROUND OF THE INVENTION

There are a wide variety of medical treatments that include the administration of a therapeutic fluid in precise, known amounts at predetermined intervals. Devices and methods exist that are directed to the delivery of such fluids, which may be liquids or gases, are known in the art.

One category of such fluid delivery devices includes insulin injecting pumps developed for administering insulin to patients afflicted with type 1, or in some cases, type 2 diabetes. Some insulin injecting pumps are configured as portable or ambulatory infusion devices that can provide continuous subcutaneous insulin injection and/or infusion therapy as an alternative to multiple daily insulin injections via syringe or injector pen. Such ambulatory infusion pumps may be worn by the user, may use replaceable medicament cartridges, and may deliver other medicaments alone, or in combination with insulin. Such medicaments include glucagon, pramlintide, and the like. Examples of such pumps and various features associated therewith include those disclosed in U.S. Patent Publication Nos. 2013/0324928 and 2013/0053816 and U.S. Pat. Nos. 8,287,495; 8,573,027; 8,986,253; and 9,381,297, each of which is incorporated herein by reference in its entirety.

Ambulatory infusion pumps for delivering insulin or other medicaments can be used in conjunction with blood glucose monitoring systems, such as continuous glucose monitoring (CGM) devices. A CGM device consists of a sensor placed under the patient's skin and affixed to the patient via an adhesive patch, a transmitter, and a monitor. A CGM device samples the patient's interstitial fluid periodically (e.g. once every 1-5 minutes) to estimate blood glucose levels over time. CGMs are advantageous because they provide more frequent insights into a user's blood glucose levels yet do not require a finger stick each time a reading is taken.

Ambulatory infusion pumps may incorporate a CGM within the hardware of the pump or may communicate with a dedicated CGM directly via a wired connection or indirectly via a wireless connection using wireless data communication protocols to communicate with a separate device (e.g., a dedicated remote device or a smartphone). One example of integration of ambulatory infusion pumps with CGM devices is described in U.S. Patent Publication No. 2014/0276419, which is hereby incorporated by reference herein. Ambulatory infusion pumps typically allow the user or caregiver to adjust the amount of insulin or other medicament delivered by a basal rate or a bolus, based on blood glucose data obtained by a CGM device, and in some cases include the capability to automatically adjust such medicament delivery. For example, based on CGM readings, some ambulatory infusion pumps may automatically adjust or prompt the user to adjust the level of medicament being administered or planned for administration or, in cases of abnormally low blood glucose readings, reducing or temporarily ceasing insulin administration.

In some cases, ambulatory insulin pumps may be configured to deliver insulin based on CGM data in a closed-loop or semi-closed-loop fashion. Some systems including these features may be referred to as automated insulin delivery (AID) systems or artificial pancreas systems because these systems serve to mimic biological functions of the pancreas for persons with diabetes.

### SUMMARY OF THE INVENTION

Disclosed herein are systems and methods for automated insulin delivery that reduce a risk of hypoglycemia from automatically delivering correction boluses. Rather than automatically delivering a correction bolus when a current or future predicted glucose level of a user is over a high glucose threshold, the system can review additional factors to determine whether an automatic correction bolus is appropriate. For example, the system can be prevented from delivering an automatic correction bolus if the user's glucose levels are falling at greater than a predetermined rate and/or if a current glucose level is greater than a future predicted glucose level even if the user's current or predicted future glucose levels is over the high threshold.

In an embodiment, an ambulatory infusion pump system includes a pump mechanism configured to facilitate delivery of insulin to a user, a communications device adapted to receive glucose levels from a continuous glucose monitor and at least one processor. The at least one processor can be configured to automatically calculate and cause correction boluses to be delivered with the pump mechanism when glucose levels of a user received from the continuous glucose monitor are over a high glucose threshold. If the at least one processor determines that delivery of an automatic correction bolus to the user with the user having a glucose level over the high glucose threshold would cause a risk of a low glucose level in the user it can prevent delivery of automatic correction boluses for a predetermined period.

In an embodiment, a method of diabetes therapy includes automatically calculating and causing correction boluses to be delivered when glucose levels of a user are over a high glucose threshold. If it is determined that delivery of an automatic correction bolus to the user with the user having a glucose level over the high glucose threshold would cause a risk of a low glucose level in the user delivery of automatic correction boluses can be prevented for a predetermined period if it is determined that delivery of an automatic correction bolus would cause a risk of a low glucose level in the user.

### BRIEF DESCRIPTION OF THE DRAWINGS

The invention may be more completely understood in consideration of the following detailed description of various embodiments of the invention in connection with the accompanying drawings, in which:

FIG. 1 is an embodiment of an ambulatory infusion pump for use with embodiments of the disclosure.

FIG. 2 is a block diagram of the ambulatory infusion pump of FIG. 1.

FIGS. 3A-3B are an alternate embodiment of an ambulatory infusion pump for use with embodiments of the disclosure.

FIG. 4 is an embodiment of a CGM for use with embodiments of the disclosure.

FIG. 5 is a schematic representation of a closed-loop insulin delivery algorithm according to the disclosure.

FIG. 6 is a graphical representation of a closed loop insulin delivery system and patient response according to an embodiment.

FIG. 7 is a flowchart of steps in a method of diabetes therapy according to the disclosure.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The following detailed description should be read with reference to the drawings in which similar elements in different drawings are numbered the same. The drawings, which are not necessarily to scale, depict illustrative embodiments and are not intended to limit the scope of the invention.

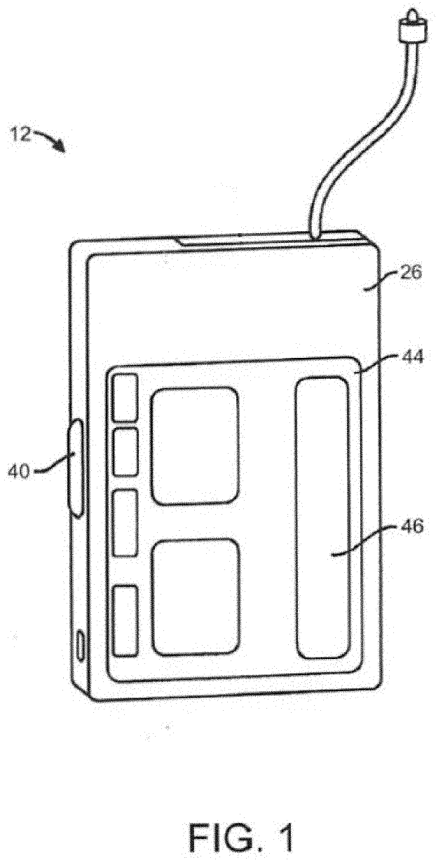

FIG. 1 depicts an example infusion pump that can be used in conjunction with one or more embodiments of the ambulatory infusion pump system of the present disclosure. Pump 12 includes a pumping or delivery mechanism and reservoir for delivering insulin or other medicament to a patient and an output/display 44. The output/display 44 may include an interactive and/or touch sensitive screen 46 having an input device such as, for example, a touch screen comprising a capacitive screen or a resistive screen. The pump 12 may additionally or instead include one or more of a keyboard, a microphone or other input devices known in the art for data entry, some or all of which may be separate from the display. The pump 12 may also include a capability to operatively couple to one or more other display devices such as a remote display (e.g., a dedicated remote display or a CGM display), a remote control device, or a consumer electronic device (e.g., laptop computer, personal computer, tablet computer, smartphone, electronic watch, electronic health or fitness monitor, or personal digital assistant). Further details regarding such pump devices can be found in U.S. Pat. No. 8,287,495, previously incorporated by reference above. It is to be appreciated that pump 12 may be optionally configured to deliver one or more additional or other medicaments to a patient.

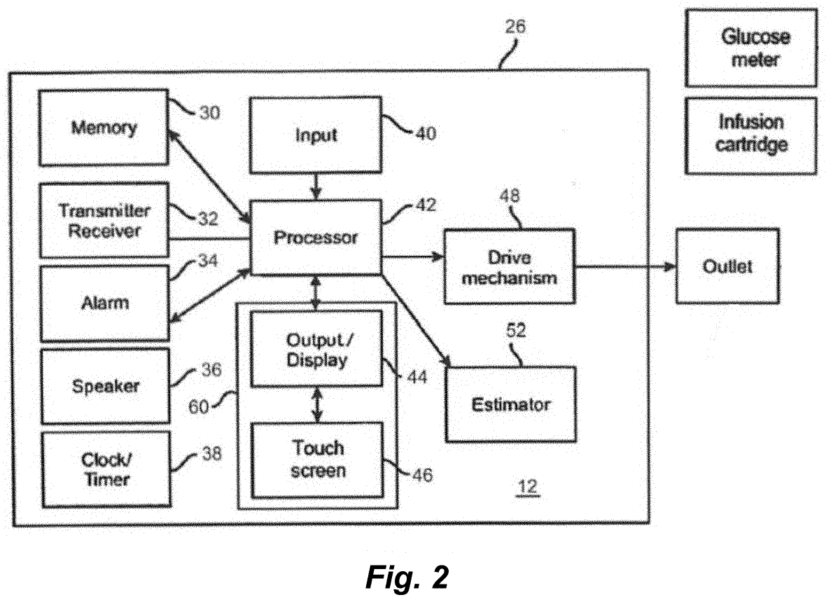

FIG. 2 illustrates a block diagram of some of the features that may be included within the housing 26 of pump 12. The pump 12 can include a processor 42 that controls the overall functions of the pump. The pump 12 may also include, e.g., a memory device 30, a transmitter/receiver 32, an alarm 34, a speaker 36, a clock/timer 38, an input device 40, a user interface suitable for accepting input and commands from a user such as a caregiver or patient, a drive mechanism 48, an estimator device 52 and a microphone (not pictured). One embodiment of a user interface is a graphical user interface (GUI) 60 having a touch sensitive screen 46 with input capability. In some embodiments, the processor 42 may communicate with one or more other processors within the pump 12 and/or one or more processors of other devices through the transmitter/receiver 32 such as a remote device (e.g., CGM device), a remote control device, or a consumer electronic device (e.g., laptop computer, personal computer, tablet computer, smartphone, electronic watch, electronic health or fitness monitor, or personal digital assistant). In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. The processor 42 may also include programming to receive signals and/or other data from an input device, such as, by way of example, a pressure sensor, a temperature sensor, or the like.

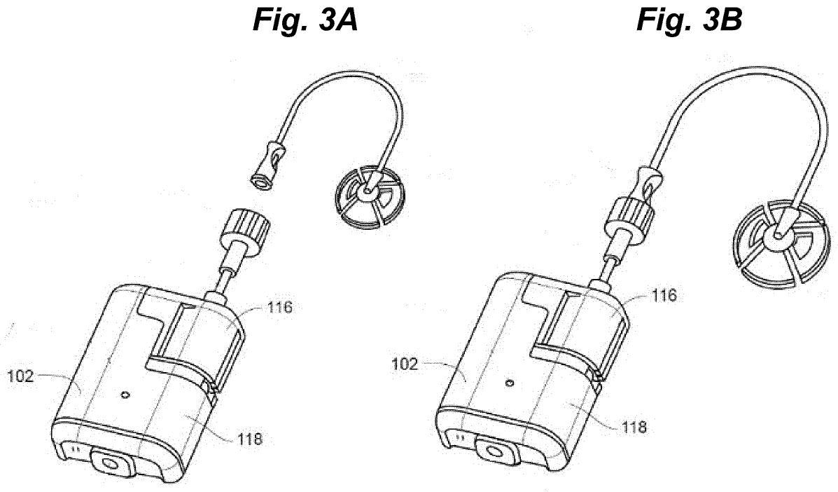

FIGS. 3A-3B depicts a second infusion pump that can be used in conjunction with one or more embodiments of the ambulatory infusion pump system of the present disclosure. Pump 102 includes a pump drive unit 118 and a medicament cartridge 116. Pump 102 includes a processor that may communicate with one or more processors within the pump 102 and/or one or more processors of other devices such as a remote device (e.g., a CGM device), a remote control device, or a consumer electronic device (e.g., laptop computer, personal computer, tablet computer, smartphone, electronic watch, electronic health or fitness monitor, or personal digital assistant). The processor 42 may also include programming to receive signals and/or other data from an input device, such as, by way of example, a pressure sensor, a temperature sensor, or the like. Pump 102 also includes a processor that controls some or all of the operations of the pump. In some embodiments, pump 102 receive commands from a separate device for control of some or all of the operations of the pump. Such separate device can include, for example, a dedicated remote control device or a consumer electronic device such as a smartphone having a processor executing an application configured to enable the device to transmit operating commands to the processor of pump 102. In some embodiments, processor can also transmit information to one or more separate devices, such as information pertaining to device parameters, alarms, reminders, pump status, etc. Such separate device can include any remote display, remote control device, or a consumer electronic device as described above. Pump 102 can also incorporate any or all of the features described with respect to pump 12 in FIG. 2. In some embodiments, the communication is effectuated wirelessly, by way of example only, via a near field communication (NFC) radio frequency (RF) transmitter or a transmitter operating according to a “Wi-Fi” or Bluetooth® protocol, Bluetooth® low energy protocol or the like. Further details regarding such pumps can be found in U.S. Pat. No. 10,279,106 and U.S. Patent Publication Nos. 2016/0339172 and 2017/0049957, each of which is hereby incorporated herein by reference in its entirety.

In some embodiments, all elements of an infusion pump system such as, e.g., the user interface, processor(s), pump mechanism, etc., reside in a single device, such as an infusion pump. In other embodiments, an infusion pump system may be a distributed system in which portions of the functionality such as, e.g., the user interface, speaker, processor, dosing algorithm, etc. may reside in separate devices such as in the infusion pump, dedicated remote control and/or other mobile device such as a mobile phone, or central computer system such as a cloud computing system.

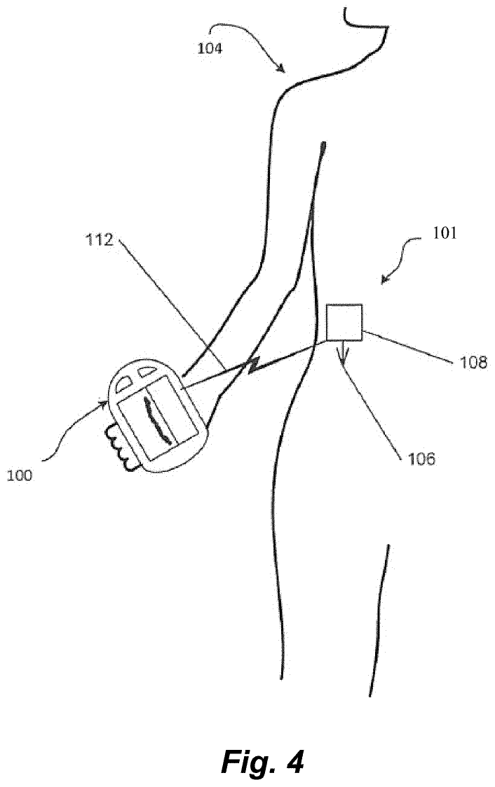

FIG. 4 depicts an example CGM system that can be used in conjunction with one or more embodiments of the ambulatory infusion pump system of the present disclosure. The CGM system includes a sensor 101, a sensor probe 106, a sensor body 108, a receiver, and a monitor (receiver and monitor are depicted as device 100 in FIG. 4). The sensor 101 is removably affixed to a user 104 and includes a sensor probe 106 configured for transcutaneous insertion into the user 104. When placed, the sensor probe 106 reacts with the user's interstitial fluid which produces a signal that can be associated with the user's blood glucose level. The sensor 101 further includes a sensor body 108 that transmits data associated with the signal to the receiver 100 via wired or wireless connection (as represented by arrow line 112). In preferred embodiments, the receiver 100 receives the transmitted data wirelessly by any suitable means of wireless communication. By way of example only, this wireless communication may include a near field communication (NFC) radio frequency (RF) transmitter or a transmitter operating according to a “Wi-Fi” or Bluetooth® protocol, Bluetooth® low energy protocol or the like. Further detail regarding such systems and definitions of related terms can be found in, e.g., U.S. Pat. Nos. 8,311,749, 7,711,402 and 7,497,827, each of which is hereby incorporated by reference in its entirety.

With the infusion pump and CGM interfaced, the CGM can automatically transmit the CGM data to the pump. The pump can then use this data to automatically determine therapy parameters and suggest a therapy adjustment to the user or automatically deliver the therapy adjustment to the user. These therapy parameters including thresholds and target values can be stored in memory located in the pump or, if not located in the pump, stored in a separate location and accessible by the pump processor (e.g., “cloud” storage, a smartphone, a CGM, a dedicated controller, a computer, etc., any of which is accessible via a network connection). The pump processor can periodically and/or continually execute instructions for a checking function that accesses these data in memory, compares them with data received from the CGM and acts accordingly to adjust therapy. In further embodiments, rather than the pump determining the therapy parameters, the parameters can be determined by a separate device and transmitted to the pump for execution. In such embodiments, a separate device such as the CGM or a device in communication with the CGM, such as, for example, a smartphone, dedicated controller, electronic tablet, computer, etc. can include a processor programmed to calculate therapy parameters based on the CGM data that then instruct the pump to provide therapy according to the calculated parameters.

For example, if the CGM readings indicate that the user has or is predicted to have a high blood glucose level, the ambulatory infusion system can automatically calculate an insulin dose sufficient to reduce the user's blood glucose level below a threshold level or to a target level and automatically deliver the dose. Alternatively, the ambulatory infusion system can automatically suggest a change in therapy upon receiving the CGM readings such as an increased insulin basal rate or delivery of a bolus, but can require the user to accept the suggested change prior to delivery rather than automatically delivering the therapy adjustments.

By way of further example, if the CGM readings indicate that the user has or is predicted to have a low blood glucose level (hypoglycemia), the ambulatory infusion system can, for example, automatically reduce or suspend a basal rate, suggest to the user to reduce a basal rate, automatically deliver or suggest that the user initiate the delivery of an amount of a substance such as, e.g., a hormone (glucagon) to raise the concentration of glucose in the blood, automatically suggest that the patient address the hypoglycemic condition as necessary (e.g., ingest carbohydrates), singly or in any desired combination or sequence.

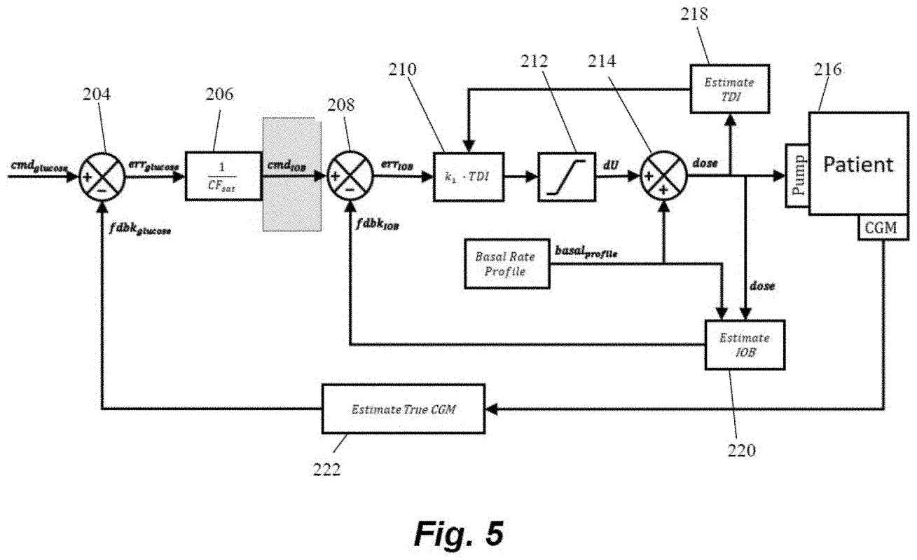

A schematic representation of a control algorithm for automatically adjusting insulin delivery based on CGM data is depicted in FIG. 5. This figure depicts an algorithm for increasing basal rate that utilizes a cascaded loop. The logic for decreasing basal rate is not depicted. In the depicted embodiment, there is a glucose set-point/command (cmd) that is determined at step 202. The glucose set point is a target value at which the algorithm attempts to maintain a user's blood glucose. This value can vary based on a number of factors, including the user's physiology, whether the user is awake or asleep, how long the user has been awake, etc. The glucose set point is compared to the actual CGM feedback (fdbk) at step 204 to determine a glucose error value (err) that is the difference between the set point and the feedback. In various embodiments, the CGM feedback can be a current glucose level reading received from a CGM or can be a predicted future glucose value based on previous glucose readings. For example, the system may predict a glucose level 30 minutes in the future (Gpred30) and utilized the predicted value as the fdbk glucose value. The errGLUCOSE value at step 206 is multiplied by a constant (1/CF), in which CF is the user's correction factor, or amount by which one unit of insulin lowers the user's blood glucose. This calculation determines how much insulin is needed to correct the glucose error, which is how much insulin on board (IOB) is needed in the user's body. This IOB value then determines an appropriate estimated insulin on board (IOB) set point for the patient.

The estimated IOB level determined at step 206 is then taken as the command (cmdIOB) for the inner loop and based on a difference of an IOB feedback value (fdbkIOB) and the cmdIOB set point at step 208, an IOB error value (errIOB) is determined. At step 210, the errIOB value is multiplied by a constant kl (relating to insulin-dependent glucose uptake in the body) and an estimate of the total daily insulin (TDI) of the user. This adjusts the errIOB to be proportional to the constant and the user's total daily intake of insulin. At step 212, a limiter function is applied to the value calculated at step 210. The limiter function can prevent the calculated amount from being larger or smaller than preset limits. The result is an insulin amount dU, which is the amount by which the user's stored basal rate should be modified. The insulin delivery rate for the user for the next closed loop interval is therefore calculated by modifying the user's stored basal rate profile by the dU value at step 214.

After the dose is calculated, it can be delivered to the user at step 216 and can also be used to update the estimated TDI for the user at step 218. The dose can also be used to update the estimated IOB level for the user at step 220 by comparing the actual insulin delivered to the programmed basal rate. The updated estimated IOB then becomes the new fdbkIOB for the IOB comparison at step 208. When new CGM values are received from the CGM, an estimated true CGM can be determined based on various factors such as, for example, the calibration status of the CGM sensor. The estimated true CGM value then becomes the new fdbkGLUCOSE value for the outer loop comparison with cmdGLUCOSE at step 204 or the estimated true CGM value can be used to update the predicted future glucose level (i.e., Gpred30) for the comparison. The algorithm then proceeds through to calculate a new estimated IOB and to the inner IOB loop for calculation of an insulin dose as described above. In one embodiment, a new CGM value is received every 5 minutes and therefore the algorithm executes as set forth above every 5 minutes.

In addition to automatically modifying basal delivery of insulin based on CGM data as described above, automated insulin delivery systems disclosed herein can also automatically deliver boluses of insulin in certain circumstances. For example, automatic correction boluses can be delivered in situations where a greater amount of insulin is needed more urgently that would be delivered with basal delivery adjustments occurring every 5 minutes. For example, basal insulin may be increased when the user's current or predicted glucose level is above a target glucose level, but if the user's glucose is above a high glucose threshold, such as, for example, 180 mg/dL an automatic correction bolus can be delivered. In embodiments, to mitigate risk of hypoglycemia from automatic correction boluses, auto-boluses can be limited by frequency and/or amount (i.e., delivered in reduced amounts relative to a full correction bolus). In one embodiment, automatic correction boluses can be given only once per hour and are delivered at 60% of a full correction bolus calculated to bring the user's glucose level to the target level.

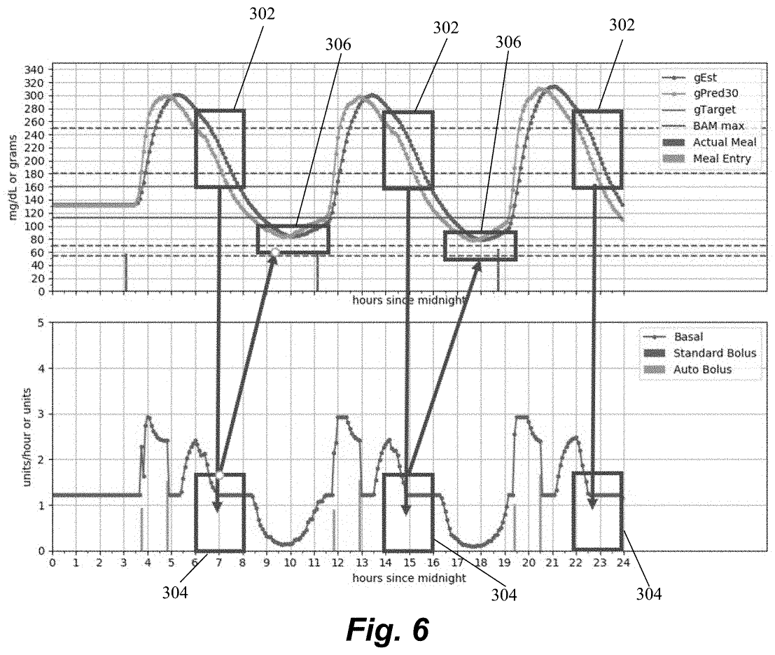

However, delivering automatic correction boluses based solely on a current or predicted glucose level being above a high threshold can potentially lead to low glucose levels in certain circumstances. Referring to FIG. 6, it can be seen that at the time periods indicated by boxes 302, the user's glucose level is above a high glucose threshold but falling rapidly. If, in response to the high glucose levels, auto-boluses are delivered during the time periods as indicated by boxes 304, the auto-boluses can cause a severe drop in glucose levels indicated in boxes 306 to near or below a low glucose threshold. Embodiments of automated insulin delivery systems disclosed herein therefore can employ additional steps to safeguard against such drops in glucose levels that could be caused by automatic correction boluses.

For example, in one embodiment the system in addition to comparing a current or predicted future glucose level, e.g., gPred30 to a high glucose threshold, the system additionally monitors a rate of change of, e.g., gPred30. For example, the system can be locked out from delivering automatic boluses that would otherwise be delivered based on gPred30 being over a threshold if gPred30 is falling at over a predetermined rate. For example, the system can be locked out from auto-boluses for, e.g., 5 minutes (i.e., until the next CGM reading) if:

$$Δ gPred 30 ( t ) \leq - 10 \frac{m g}{dL} , \\ where Δ gPred 30 ( t ) = g P r e d 30 ( t ) - gPred 30 ( t - 15 ) , \qquad ( 3 )$$

with t in minutes. In other words, if gPred30 has dropped by 10 mg/dL or more over the last 15 minutes, the system is prevented from delivering an automatic bolus for a predetermined time period even if gPred30 is over the high threshold. Thus, at any given time t, the system will look back a previous number of CGM readings to determine a rate of change of the user's glucose levels over that time and will prevent automatic correction boluses if the rate of change is falling over a predetermined amount. After the temporary lockout, the system can again utilize the above equation to determine whether the system can deliver automatic correction boluses upon receiving the next value from the CGM. All of the threshold rate of change, time over which the change is calculated and time that the system is locked out from delivering auto-boluses may vary.

Alternatively, in an embodiment the system can compare a predicted future glucose level such as gPred30 to a current glucose level estimate (gEst) to determine whether automatic boluses can be delivered. For example, if gEst>gPred30 for a current glucose level estimate the system can be prevented from delivering automatic boluses until the next glucose level estimate (i.e., five minutes), until a certain number of glucose level estimates, etc. This is because if the current glucose level estimate is greater than the future predicted glucose level, it means that the user's glucose level is already falling and may continue to fall below the high threshold even if the gPred30 estimate is over the threshold. The comparison can be conducted again after the lockout period.

The time for which automatic correction boluses are prevented from being delivered can vary based on an amount of time or a number of CGM readings. For example, the auto-boluses may be locked out until the next CGM value is received or until a certain number of subsequent CGM values are received. Alternatively, the system may be locked out from auto-boluses for a predetermined period of time.

In another embodiment, a bolus lockout feature as described above can be employed after a low glucose alert indicating that a user's glucose levels have dropped below a low glucose threshold is issued. Some low glucose alerts instruct a user to ingest carbohydrates in order to raise the user's glucose level and, regardless of the specific content of the alert, many diabetic user's response to such alerts by eating to ingest “rescue carbs” to raise the user's glucose levels. Ingestion of such rescue carbs can cause a quick upward rise in glucose levels, which can cause some closed loops systems to project a future glucose level such as gpred30 higher than it will actually become and to therefore generate an automatic correction bolus. Such an automatic correction bolus would counteract the rescue carbs and potentially lead to the user's glucose levels going low again and/or to undesirable blood glucose oscillations. As such, embodiments of the present disclosure can employ a bolus lockout feature that prevents delivery of automatic correction boluses for a predetermined time following detection of a low glucose level and/or delivery of an alert informing the user of such a low glucose level. In various embodiments, the predetermined time can be, e.g., 30 minutes, 45 minutes, and hour etc., so long as it is sufficient time to enable the user's glucose levels to stabilize following ingestion of any rescue carbs the user might have taken following the alert.

In some embodiments, the approaches described above are only applied within certain glucose level ranges. If the user's glucose level is extremely high an automatic correction bolus may be needed and would be unlikely to cause hypoglycemia even if the current or future predicted glucose levels are dropping rapidly and/or the current glucose level estimate is greater that the future predicted glucose level. In contrast, if the user's current or predicted glucose level is over but relatively near to the high threshold and/or is dropping extremely rapidly, an automatic correction bolus may increase the risk of hypoglycemia. For example, if the user's current glucose level estimate is 400 mg/dL and gPred30 is 398 mg/dL, the user's glucose level is dropping, but an automatic correction bolus would be unlikely to drop the user's glucose level below a low glucose threshold such as, e.g., 70 mg/dL. However, if the user's current glucose level is must closer to the high threshold and is dropping, e.g., a current glucose estimate of 225 mg/dL and a gPred30 of 200 mg/dL, the risk of hypoglycemia in response to a bolus to further lower glucose levels is increased and an automatic bolus lockout may be appropriate. As such, in some embodiments the auto-bolus lockout feature is employed only when the current and/or predicted future glucose level of the user is within a predetermined amount of the high threshold (or below a second, higher threshold) and is disabled if the glucose level is above the predetermined amount (or above the second, higher threshold). In one embodiment, the lockout is disabled if the current or future predicted glucose level is greater than or equal to 250 mg/dL (i.e., severe hyperglycemia).

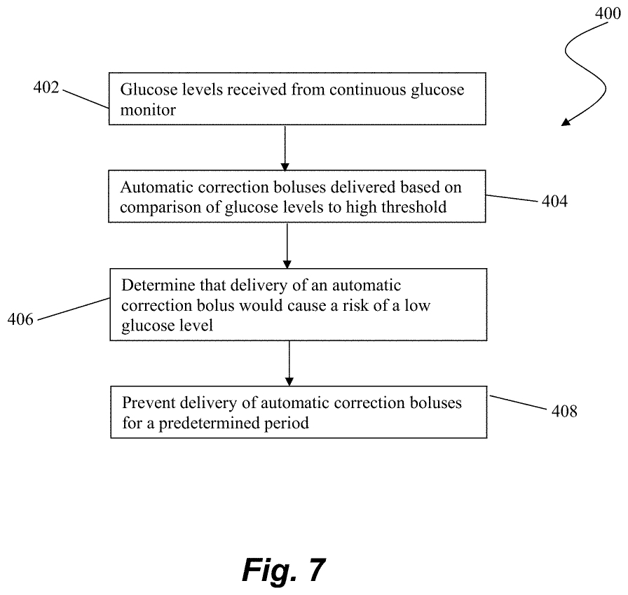

FIG. 7 is a flowchart of steps in a method of diabetes therapy 400 according to the disclosure. At step 402, glucose levels of a user are received from a continuous glucose monitor. Automatic correction boluses can then be delivered as needed based on the glucose levels by comparing the glucose levels to a high glucose threshold at step 404. It is determined at step 406 that delivery of an automatic correction bolus would cause a risk of a low glucose level. This can be determined, for example, based on a rate of change of the user's glucose levels, comparing a future predicted glucose level of the user to a current glucose level, based on a recent low glucose notification, etc. In response to the determination, at step 408 the system can be prevented from delivering automatic correction boluses for a predetermined period.

In embodiments, an ambulatory infusion pump system includes a pump mechanism configured to facilitate delivery of insulin to a user, a communications device adapted to receive glucose levels from a continuous glucose monitor and at least one processor. The at least one processor can be configured to automatically calculate and cause correction boluses to be delivered with the pump mechanism when glucose levels of a user received from the continuous glucose monitor are over a high glucose threshold. If the at least one processor determines that delivery of an automatic correction bolus to the user with the user having a glucose level over the high glucose threshold would cause a risk of a low glucose level in the user it can prevent delivery of automatic correction boluses for a predetermined period.

In some embodiments, the at least one processor can be configured to determine that delivery of an automatic correction bolus would cause a risk of a low glucose level in the user if a rate of change of the user's glucose levels is falling at more than a predetermined rate.

In some embodiments, the at least one processor can be configured to determine the rate of change over a predetermined period of time.

In some embodiments, the at least one processor can be configured to determine that delivery of an automatic correction bolus would cause a risk of a low glucose level in the user if a current glucose level of the user is greater than a predicted future glucose level of the user.

In some embodiments, the at least one processor can be configured to determine that delivery of an automatic correction bolus would cause a risk of a low glucose level in the user if a low glucose alert indicating that a glucose level of a user is below a low threshold had been issued within a predetermined period of time.

In some embodiments, the glucose level of the user can be a current glucose level.

In some embodiments, the glucose level of the user can be a predicted future glucose level based on the glucose levels from the continuous glucose monitor.

In some embodiments, the predetermined period can be a predetermined amount of time.

In some embodiments, the predetermined period can end upon receipt of a subsequent glucose level of the user from the continuous glucose monitor.

In embodiments, a method of diabetes therapy includes automatically calculating and causing correction boluses to be delivered when glucose levels of a user are over a high glucose threshold. If it is determined that delivery of an automatic correction bolus to the user with the user having a glucose level over the high glucose threshold would cause a risk of a low glucose level in the user delivery of automatic correction boluses can be prevented for a predetermined period if it is determined that delivery of an automatic correction bolus would cause a risk of a low glucose level in the user.

In some embodiments, it can be determined that delivery of an automatic correction bolus would cause a risk of a low glucose level in the user if a rate of change of the user's glucose levels is falling at more than a predetermined rate.

In some embodiments, the rate of change can be determined over a predetermined period of time.

In some embodiments, it can be determined that delivery of an automatic correction bolus would cause a risk of a low glucose level in the user if a current glucose level of the user is greater than a predicted future glucose level of the user.

In some embodiments, it can be determined that delivery of an automatic correction bolus would cause a risk of a low glucose level in the user if a low glucose alert indicating that a glucose level of a user is below a low threshold had been issued within a predetermined period of time.

In some embodiments, the glucose level of the user can be a current glucose level.

In some embodiments, the glucose level of the user can be a predicted future glucose level based on the glucose levels from the continuous glucose monitor.

In some embodiments, the predetermined period can be a predetermined amount of time.

In some embodiments, the predetermined period can end upon receipt of a subsequent glucose level of the user from the continuous glucose monitor.

In some embodiments, delivery of automatic correction boluses can be prevented for the predetermined period if it is determined that delivery of an automatic correction bolus would cause a risk of a low glucose level in the user and the glucose level is within a predetermined amount of the high glucose threshold.

Although embodiments described herein may be discussed in the context of the controlled delivery of insulin, delivery of other medicaments, singly or in combination with one another or with insulin, including, for example, glucagon, pramlintide, etc., as well as other applications are also contemplated. Device and method embodiments discussed herein may be used for pain medication, chemotherapy, iron chelation, immunoglobulin treatment, dextrose or saline IV delivery, treatment of various conditions including, e.g., pulmonary hypertension, or any other suitable indication or application. Non-medical applications are also contemplated.

With regard to the above detailed description, like reference numerals used therein may refer to like elements that may have the same or similar dimensions, materials, and configurations. While particular forms of embodiments have been illustrated and described, it will be apparent that various modifications can be made without departing from the spirit and scope of the embodiments herein. Accordingly, it is not intended that the invention be limited by the forgoing detailed description.

The entirety of each patent, patent application, publication, and document referenced herein is hereby incorporated by reference. Citation of the above patents, patent applications, publications and documents is not an admission that any of the foregoing is pertinent prior art, nor does it constitute any admission as to the contents or date of these documents.

Also incorporated herein by reference in their entirety are commonly owned U.S. Pat. Nos. 6,999,854; 8,133,197; 8,287,495; 8,408,421 8,448,824; 8,573,027; 8,650,937; 8,986,523; 9,173,998; 9,180,242; 9,180,243; 9,238,100; 9,242,043; 9,335,910; 9,381,271; 9,421,329; 9,486,171; 9,486,571; 9,492,608; 9,503,526; 9,555,186; 9,565,718; 9,603,995; 9,669,160; 9,715,327; 9,737,656; 9,750,871; 9,867,937; 9,867,953; 9,940,441; 9,993,595; 10,016,561; 10,201,656; 10,279,105; 10,279,106; 10,279,107; 10,357,603; 10,357,606; 10,492,141; 10,541,987; 10,569,016; 10,736,037; 10,888,655; 10,994,077; 11,116,901; and 11,224,693 and commonly owned U.S. Patent Publication Nos. 2009/0287180; 2012/0123230; 2013/0053816; 2014/0276423; 2014/0276569; 2014/0276570; 2018/0071454; 2019/0240398; 2019/0307952; 2020/0206420; 2020/0261649; 2020/0306445; 2020/0329433; 2020/0368430; 2020/0372995; 2021/0001044; 2021/0113766; 2021/0154405; and 2021/0353857 and commonly owned U.S. patent application Ser. Nos. 17/368,968; 17/459,129; 17/517,885 and 17/573,705.

Modifications may be made to the foregoing embodiments without departing from the basic aspects of the technology. Although the technology may have been described in substantial detail with reference to one or more specific embodiments, changes may be made to the embodiments specifically disclosed in this application, yet these modifications and improvements are within the scope and spirit of the technology. The technology illustratively described herein may suitably be practiced in the absence of any element(s) not specifically disclosed herein. The terms and expressions which have been employed are used as terms of description and not of limitation and use of such terms and expressions do not exclude any equivalents of the features shown and described or portions thereof and various modifications are possible within the scope of the technology claimed. Although the present technology has been specifically disclosed by representative embodiments and optional features, modification and variation of the concepts herein disclosed may be made, and such modifications and variations may be considered within the scope of this technology.

1. An ambulatory infusion pump system, comprising:
a pump mechanism configured to facilitate delivery of insulin to a user;
a communications interface adapted to receive glucose levels from a continuous glucose monitor; and
at least one processor configured to:
automatically calculate and cause correction boluses to be delivered with the pump mechanism when glucose levels of the user received from the continuous glucose monitor are over a high glucose threshold;
determine that delivery of an automatic correction bolus to the user with the user having a glucose level over the high glucose threshold would cause a risk of a low glucose level in the user; and
prevent delivery of automatic correction boluses for a predetermined period of time if it is determined that delivery of an automatic correction bolus would cause a risk of a low glucose level in the user.

2. The ambulatory infusion pump system of claim 1, wherein the at least one processor is configured to determine that delivery of an automatic correction bolus would cause a risk of a low glucose level in the user if a rate of change of the user's glucose levels is falling at more than a predetermined rate.

3. The ambulatory infusion pump system of claim 1, wherein the at least one processor is configured to determine the rate of change over a predetermined period of time.

4. The ambulatory infusion pump system of claim 1, wherein the at least one processor is configured to determine that delivery of an automatic correction bolus would cause a risk of a low glucose level in the user if a current glucose level of the user is greater than a predicted future glucose level of the user.

5. The ambulatory infusion pump system of claim 1, wherein the at least one processor is configured to determine that delivery of an automatic correction bolus would cause a risk of a low glucose level in the user if a low glucose alert indicating that a glucose level of a user is below a low threshold had been issued within a predetermined period of time.

6. The ambulatory infusion pump system of claim 1, wherein the glucose level of the user is a current glucose level.

7. The ambulatory infusion pump system of claim 1, wherein the glucose level of the user is a predicted future glucose level based on the glucose levels from the continuous glucose monitor.

8. The ambulatory infusion pump system of claim 1, wherein the predetermined period is a predetermined amount of time.

9. The ambulatory infusion pump system of claim 1, wherein the predetermined period of time ends upon receipt of a subsequent glucose level of the user from the continuous glucose monitor.

10. The ambulatory infusion pump system of claim 1, wherein the at least one processor is further configured to prevent delivery of automatic correction boluses for the predetermined period of time if it is determined that delivery of an automatic correction bolus would cause a risk of a low glucose level in the user and the glucose level is within a predetermined amount of the high glucose threshold.

11. A method of diabetes therapy, comprising:
automatically calculating and causing correction boluses to be delivered when glucose levels of a user are over a high glucose threshold;
determining that delivery of an automatic correction bolus to the user with the user having a glucose level over the high glucose threshold would cause a risk of a low glucose level in the user; and
preventing delivery of automatic correction boluses for a predetermined period of time if it is determined that delivery of an automatic correction bolus would cause a risk of a low glucose level in the user.

12. The method of claim 11, wherein it is determined that delivery of an automatic correction bolus would cause a risk of a low glucose level in the user if a rate of change of the user's glucose levels is falling at more than a predetermined rate.

13. The method of claim 11, wherein the rate of change is determined over a predetermined period of time.

14. The method of claim 11, wherein it is determined that delivery of an automatic correction bolus would cause a risk of a low glucose level in the user if a current glucose level of the user is greater than a predicted future glucose level of the user.

15. The method of claim 11, wherein it is determined that delivery of an automatic correction bolus would cause a risk of a low glucose level in the user if a low glucose alert indicating that a glucose level of a user is below a low threshold had been issued within a predetermined period of time.

16. The method of claim 11, wherein the glucose level of the user is a current glucose level.

17. The method of claim 11, wherein the glucose level of the user is a predicted future glucose level based on the glucose levels from the continuous glucose monitor.

18. The method of claim 11, wherein the predetermined period is a predetermined amount of time.

19. The method of claim 11, wherein the predetermined period of time ends upon receipt of a subsequent glucose level of the user from the continuous glucose monitor.

20. The ambulatory infusion pump system of claim 11, wherein delivery of automatic correction boluses are prevented for the predetermined period of time if it is determined that delivery of an automatic correction bolus would cause a risk of a low glucose level in the user and the glucose level is within a predetermined amount of the high glucose threshold.
